(19) United States
(12) Patent Application Publication
(10) Pub. No.: US 2009/0181139 A1
(43) Pub. Date: Jul. 16, 2009

(54) Pressure Assisted Thermal Sterilisation or Pasteurisation Method and Apparatus

(76) Inventor: Mohammed Mehid Farid, Auckland (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/296,789

(22) PCT Filed: Apr. 10, 2006
(86) PCT No.: PCT/NZ2006/000069

§ 371 Date: Feb. 12, 2009

(30) Foreign Application Priority Data
Apr. 12, 2005 (NZ) 539438

(51) Int. Cl. A23L 3/10; A61L 2/04

(52) U.S. Cl. 426/234; 426/521; 422/1; 422/307

(57) ABSTRACT

The invention relates to a method suitable for pasteurisation or sterilisation of articles, and apparatus suitable for carrying out the method. The method includes heating the article and/or a related medium within a confined volume sufficiently that expansion of the article and/or the medium subjects the article to a pressure sufficient (in combination with the temperature) to result in pasteurisation or sterilisation of the article.

### FIELD OF THE INVENTION

The invention relates to the sterilisation or pasteurisation of articles such as food products utilising a combination of elevated pressure and temperature, and to a sterilisation or pasteurisation apparatus.

### BACKGROUND

High pressure processing (HPP) of food is a non-thermal sterilisation or pasteurisation process, which retains the food quality of freshness and reduces damage to nutrients such as vitamins. HPP processing can be used not only for preservation but also for changing the physical and functional properties of foods. HPP is used for treating food products which are currently available in the market including juices, jams, jellies, yogurts, meat, and seafood such as oysters.

The pressure needed to kill spores is very high. When pressure alone is used it is generally above 600 MPa, making HPP expensive to apply for complete sterilization. However, it has also been shown that spores can be killed effectively by applying moderate pressure combined with heat to ensure both quality and safety (Gould, 1969; Ananta et al, 2001; Knorr et al., 1995; Patterson et al., 1995; Rovere et al., 1996; Ludwig et al., 1992; Stewart et al., 2000; Earnshaw et al., 1995; Furukawa and Hayakawa, 2000 and Hayakawa et al., 1994). Existing technology in this area pressurises the food in combination with mild heating prior to or after pressurising the food. This sterilises or pasteurises the food product with minimum damage to the physical appearance and nutritional value of the food.

It has been noted throughout the literature, that B. stearothermophilus, one of the most (if not the most) heat resistant bacterial spores, cannot be inactivated by moderate temperature or high pressure alone. It has been noted that a particular temperature is required for bacterial spores to activate first into a germinative form before they can be inactivated by pressure. For B. stearothermophilus a range of about 49°-55° C. is generally proposed (Lund, 1975).

### OBJECT OF THE INVENTION

It is an object of the invention to provide an improved or at least alternative method and apparatus for sterilisation or pasteurisation of products, particularly food products, or at least to provide the public with a useful alternative.

### SUMMARY OF INVENTION

In broad terms in a first aspect the invention comprises a method for pasteurising or sterilising an article, comprising the step of heating the article and/or medium associated or in communication with the article within a confined volume sufficiently that expansion of the article and/or of the medium subjects the article to a pressure sufficient when combined with the elevated temperature to pasteurise or sterilise the article.

Preferably the elevated temperature is less than that required to pasteurise or sterilise the article in the absence of the pressure. Alternatively the duration of exposure of the article to the elevated temperature is less than that required to pasteurise of sterilise the article in the absence of the pressure. Alternatively both the elevated temperature and the duration of exposure of the article to the elevated temperature are less than that required to pasteurise of sterilise the article in the absence of the pressure.

In a first embodiment the article will be a food product. Where the article is a food product it is preferably heated to a temperature below about 140° C., (usually used in a short time-high temperature sterilization process) more preferably below 120° C., (usually employed in the in-can sterilization) or at normal sterilization temperature but for shorter time to minimise damage to the product and maximise retention of food quality which may otherwise occur.

In one form of the first embodiment it may be in liquid form such as a juice, milk, soup, or honey, for example, in a semi-liquid form such as a pulp or pasty product. In another form it may be in solid form such as meat for example.

An advantage of the method of the invention when used for pasteurising or sterilising food products, is that it is possible to pasteurise or sterilise products at relatively lower temperatures and/or for shorter treatment time, reducing the likelihood of thermal damage to vitamins or other nutrients in the food products which may otherwise occur at higher temperatures.

In a second embodiment the article is not a food product. It may be a pharmaceutical or medical product, biological material, or any other article benefiting from sterilisation or pasteurisation.

Thermal expansion of the article itself when heated within a confined volume may generate sufficient pressure, combined with temperature, to sterilise or pasteurise the article. Another medium (an “expansion medium”) with a co-efficient of thermal expansion typically higher than that of the article being processed may be included to generate or increase pressure on the article when heated. Such an expansion medium may be distributed throughout a liquid article, for example, as particles within an elastic covering or outer layer, which are mixed with the article before subjecting to pressure and heat and which in turn may be separated from the article after heat processing.

Alternatively the article to be treated may be contained within a first part of the confined volume and an expansion medium contained within a second part of the confined volume separated by a diaphragm or other means which will transfer pressure induced by expansion of the expansion medium to the article being treated, such as a piston arrangement for example. This configuration enables the expansion medium to be heated to a higher temperature than for example 120° C. 100° C., to maximise thermal expansion of the expansion medium and pressure on the article, without at the same time elevating the temperature to which the article is subjected to the same higher temperature, so that the temperature to which for example a food product is subjected can be kept below 120° C. or 100° C. Alternatively this will allow subjecting the product to the normal sterilisation temperature but for a shorter treatment time.

Heating of the article may be achieved by isolating the article within a heat exchanger having an outer jacket through which a heating medium such as hot water, steam or oil or similar is passed to heat the article within the heat exchanger. Alternatively, heating may be by electric resistance heating of a chamber within which the article is contained, by microwave heating, by solar or waste heat, or by any other suitable heating system.

Typically the method of the invention in relation to the sterilisation or pasteurisation of food products may be applied at a stage in a food processing plant to treat the food product in bulk form immediately prior to passing of the food product to a packaging stage of a food processing line.

In broad terms in another aspect the invention comprises an article pasteurised or sterilised according to the above method.

In broad terms in another aspect the invention comprises apparatus for sterilising or pasteurising an article, including a processing chamber providing a confined volume for containing an article to be processed, heating means for heating the article, and wherein the processing chamber is configured such that heating the article and/or an expansion medium associated or in communication with the article generates a pressure applied to the article sufficient when combined with the elevated temperature to pasteurise or sterilise the article.

Preferably the apparatus includes a control system arranged to heat the article sufficiently that expansion of the article and/or of another expansion medium subjects the article to a pressure sufficient when combined with the elevated temperature to pasteurise or sterilise the article.

In broad terms in another aspect the invention comprises a method for sterilising or pasteurising an article substantially as herein described with reference to any one or more of the examples or drawings.

In broad terms in another aspect the invention comprises apparatus for sterilising or pasteurising an article substantially as herein described with reference to any one or more of the examples or drawings.

### DEFINITIONS

As used herein the term “article” means any item which may benefit from heat/pressure treatment, particularly sterilisation or pasteurisation. It may include foodstuffs and food products, both solid and liquid phase. It will include “semi-continuous” food stuffs just as pastes or juices which may flow through a process. It will also include non-food items or products such as pharmaceutical or medical products and biological materials, for example.

As used herein the term “sterilising” means substantially complete inactivation of thermophilic spores.

As used herein the term “pasteurising” means inactivation of pathogens, often in the form of vegetative bacteria.

As used herein “food”, “food product”, or “foodstuffs” includes solid or liquid food or semi-solid foods including drinks such as juices or milk liquid drinks, and pastes.

It also includes solid food such as meat, seafood such as shell fish, and fruit As used herein the term “and/or” means “and” or “or”, or both.

As used herein “(s)” following a noun means the plural and/or singular forms of the noun.

“The term “comprising” as used in this specification and claims means “consisting at least in part of”; that is to say when interpreting statements in this specification and claims which include “comprising”, features, other than those prefaced by this term in each statement, can also be present. Related terms such as “comprise” and “comprised” are to be interpreted in similar manner.”

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention may become apparent from the following description which is given by way of example only and with reference to the accompanying drawings.

### DETAILED DESCRIPTION OF THE DRAWINGS

The invention will now be described with reference to the Figures in which:

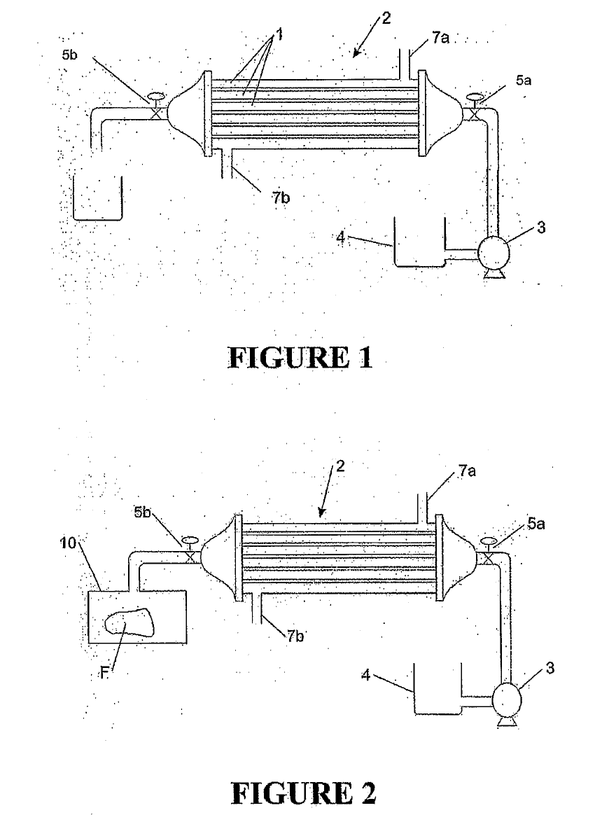

FIG. 1: a schematic of one implementation of the invention;

FIG. 2: a schematic of a further implementation of the invention;

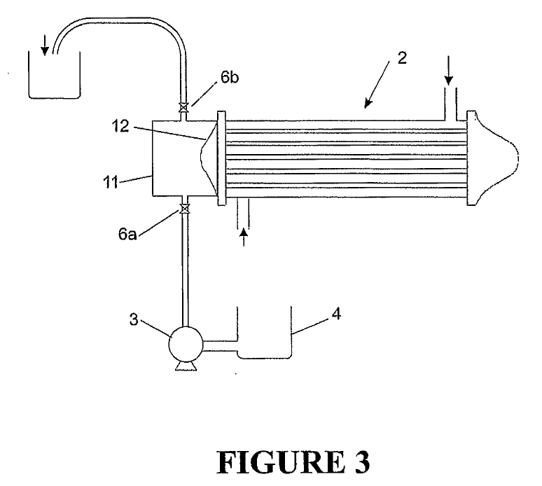

FIG. 3: a schematic of a further implementation of the invention;

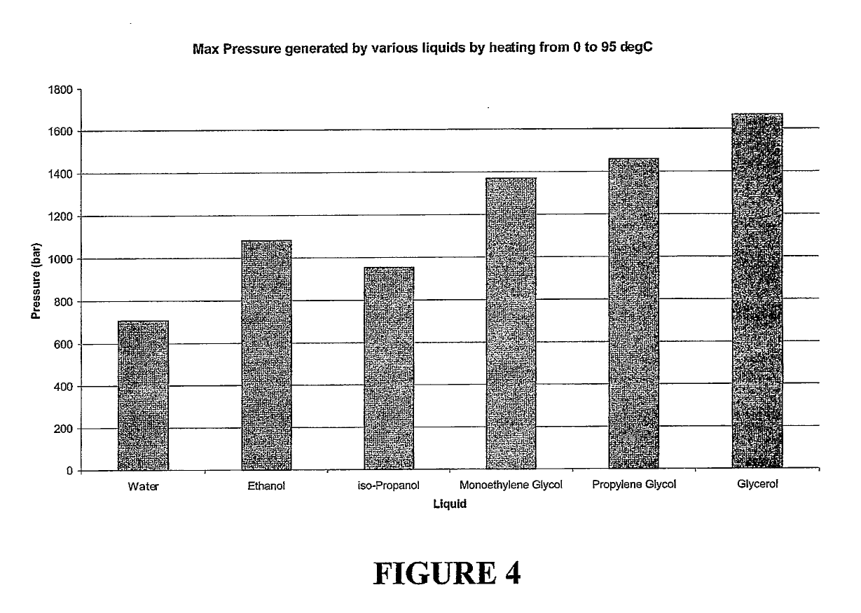

FIG. 4: a graph of the pressure generated by various liquids by heating;

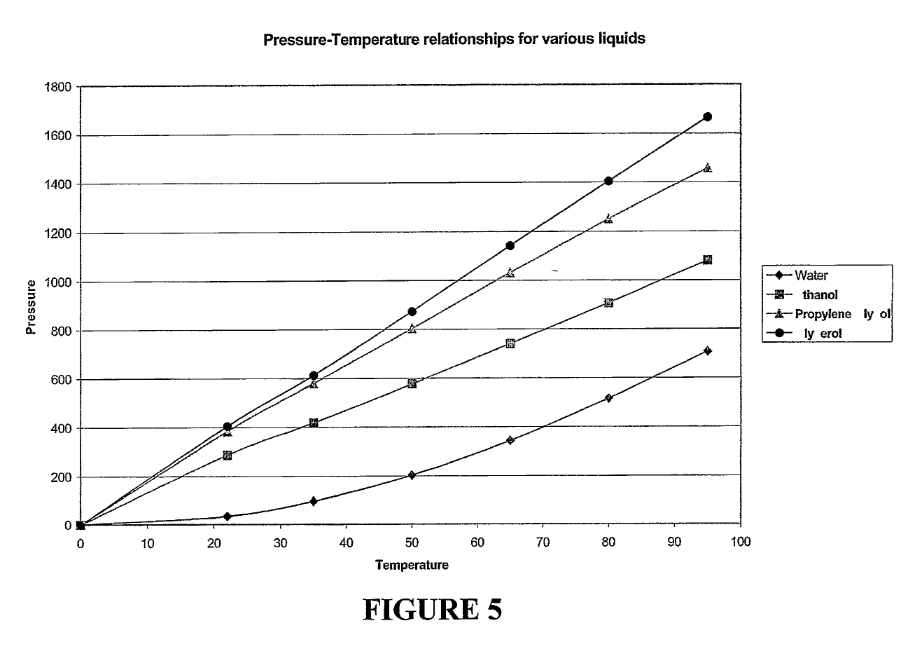

FIG. 5: a graph of pressure-temperature relationships for various liquids;

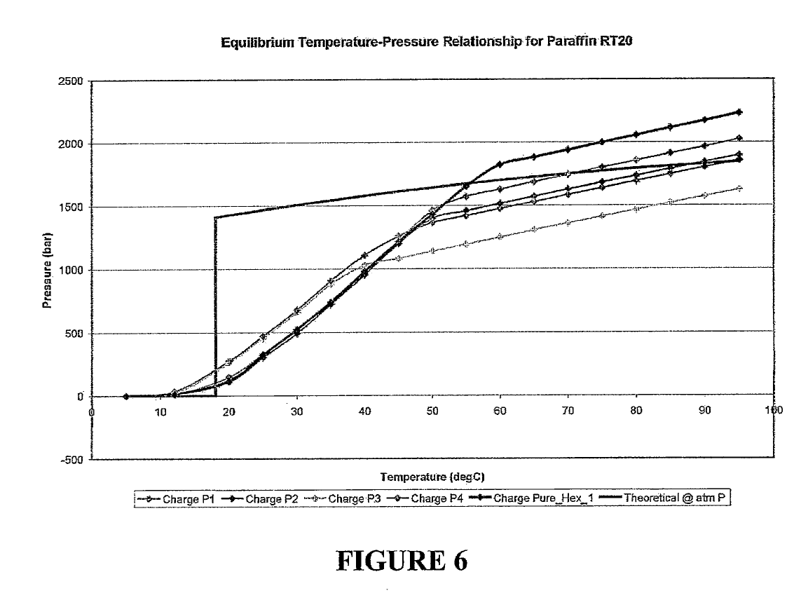

FIG. 6: a graph of the equilibrium temperature-pressure relationship for paraffin wax;

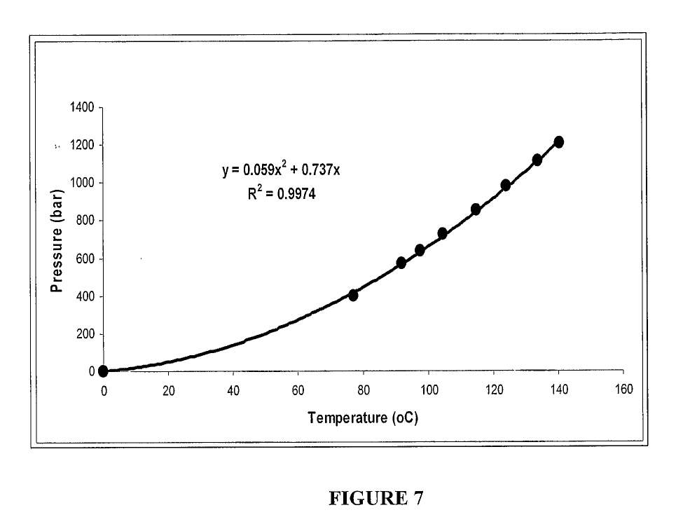

FIG. 7: a graph of the pressure-temperature equilibrium relationship for water in a heat exchanger in one implementation of the invention;

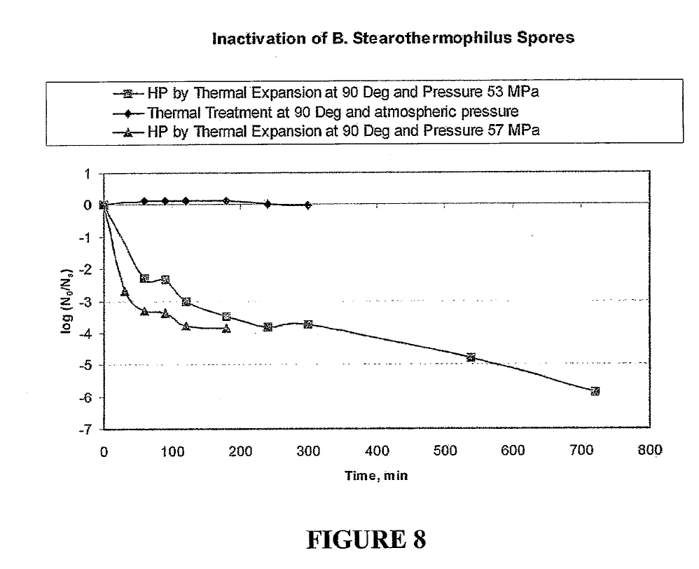

FIG. 8: a graph of bacterial spore (B. stearothermophilus) results with combined temperature/pressure treatment at 90° C.;

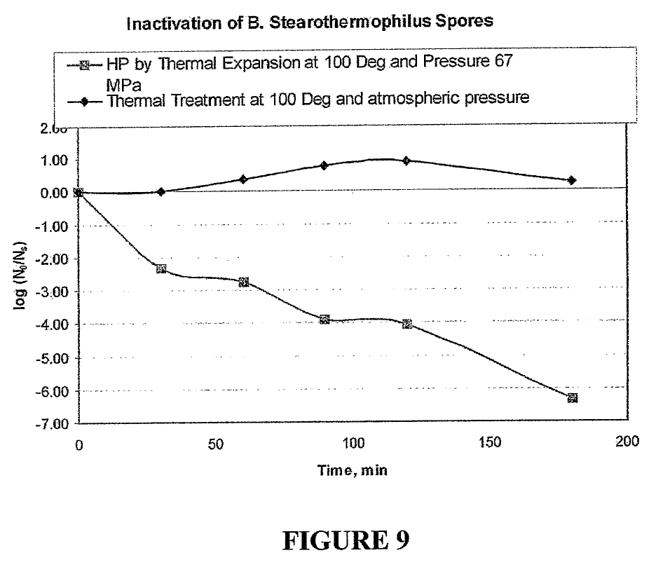

FIG. 9: a graph of bacterial spore (B. stearothermophilus) results with combined temperature/pressure treatment at 100° C.;

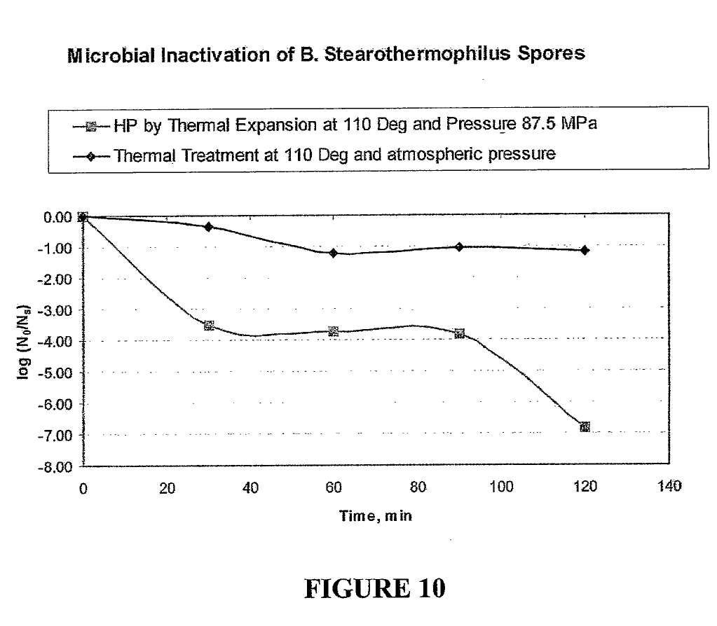

FIG. 10: a graph of bacterial spore (B. stearothermophilus) results with combined temperature/pressure treatment at 110° C.;

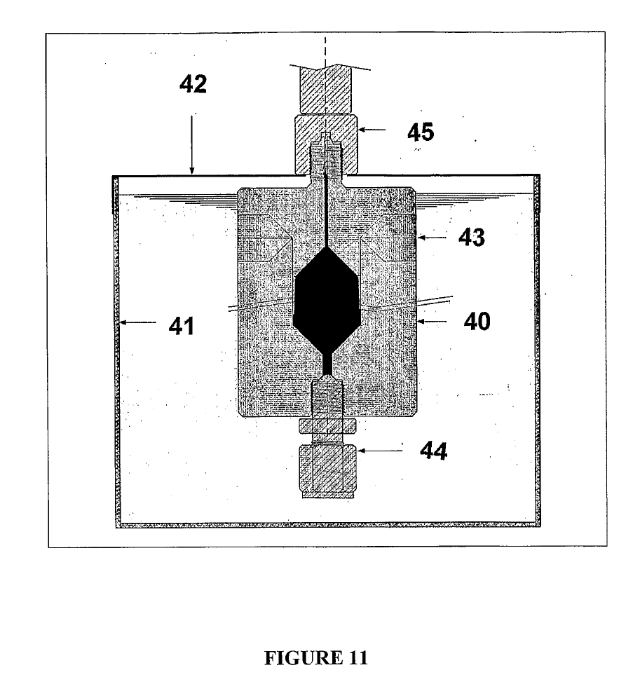

FIG. 11: a schematic view of an experimental test unit;

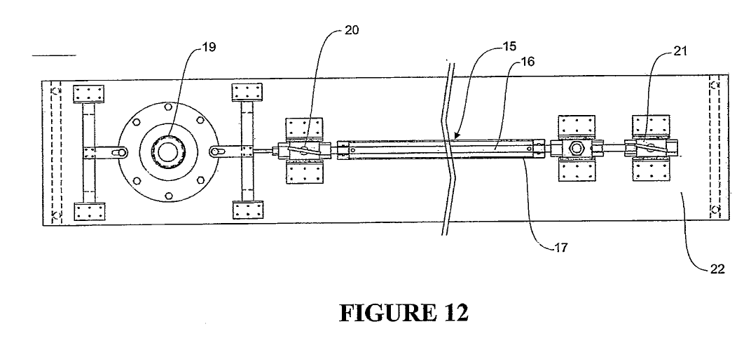

FIG. 12: a schematic of the experimental test unit (one implementation of the invention);

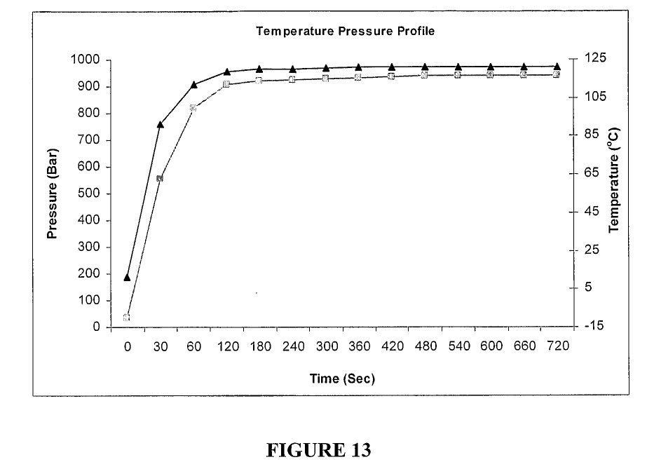

FIG. 13: a graph showing the transient temperature-pressure relationship for the high pressure implementation of the invention;

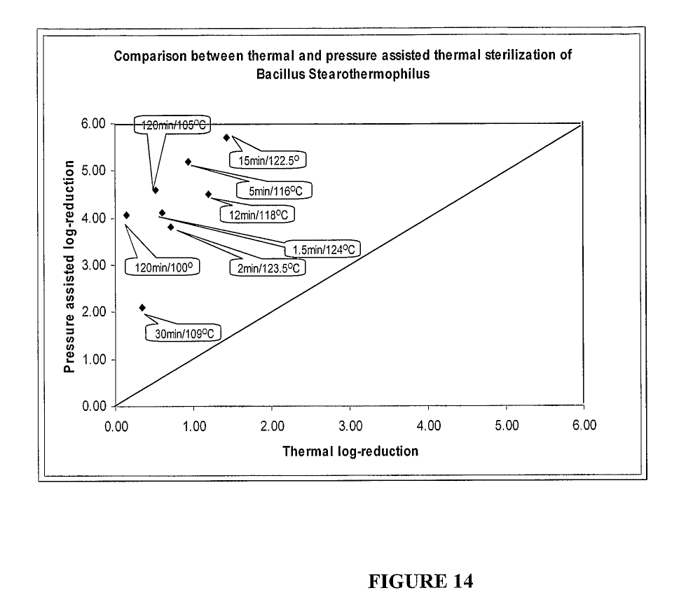

FIG. 14: a graph showing the comparison of inactivation of B. stearothermophilus spores in water using thermal and pressure assisted thermal sterilisation;

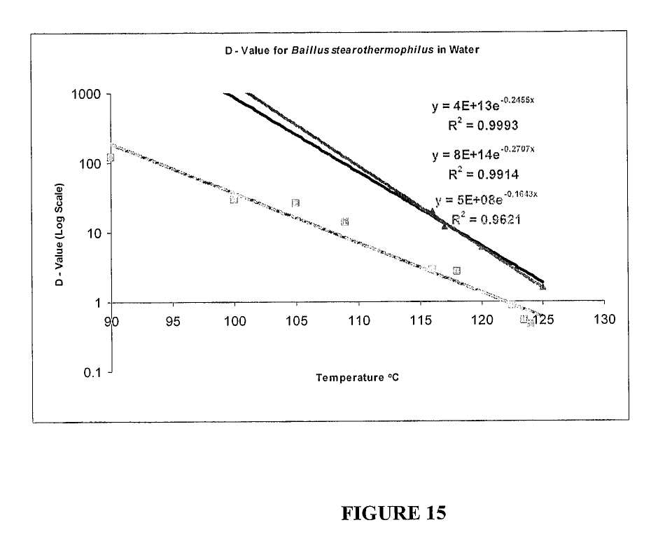

FIG. 15: a graph showing the comparison of inactivation of B. stearothermophilus spores (Decimal reduction time) in water using thermal and pressure assisted thermal sterilisation;

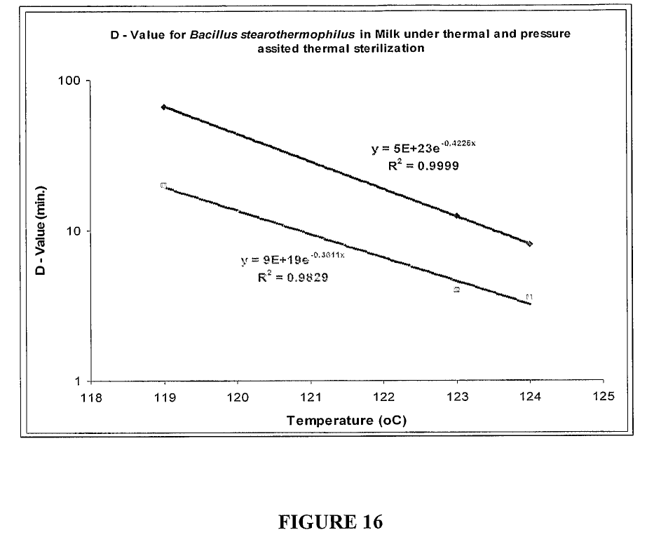

FIG. 16: a graph showing the comparison of inactivation of B. stearothermophilus spores (Decimal reduction time) in milk using thermal and pressure assisted thermal sterilisation;

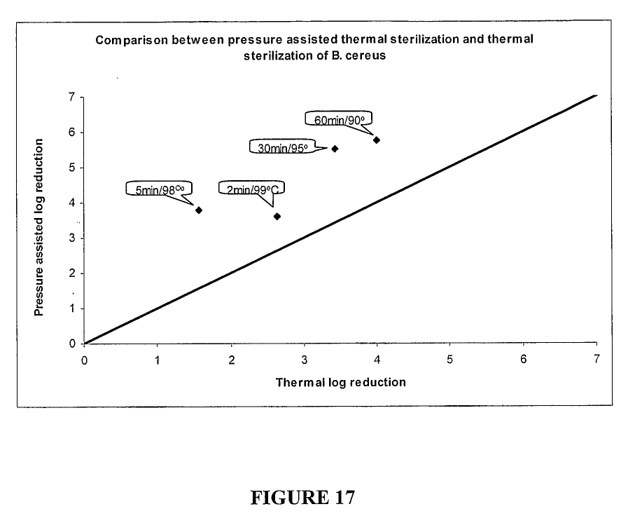

FIG. 17 a graph showing the comparison of inactivation of B. Cerus spores in water using thermal and pressure assisted thermal sterilisation;

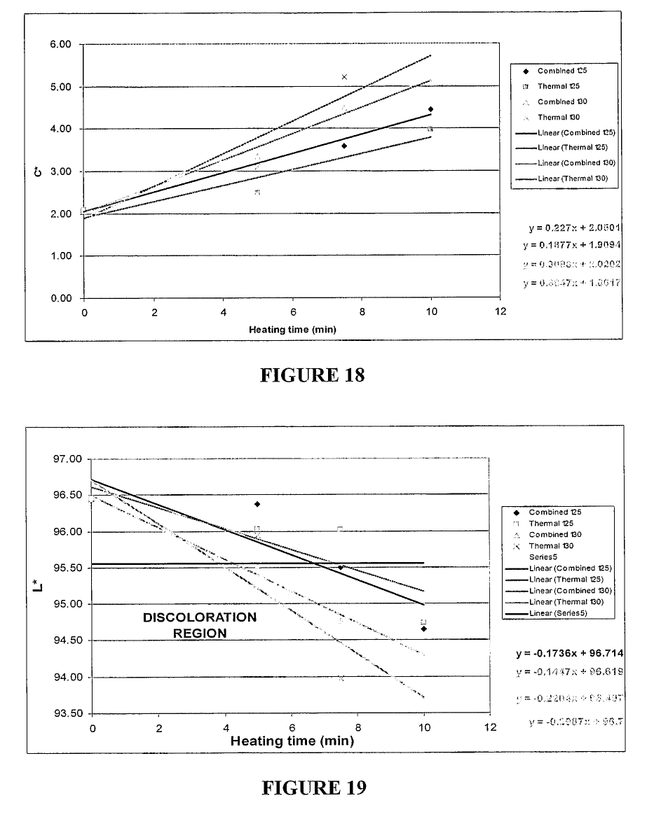

FIG. 18: a graph showing the change in Chroma value (C*) during thermal and pressure assisted thermal sterilization for results of Example 6;

FIG. 19: a graph showing the change in Luminence value (L*) during thermal and pressure assisted thermal sterilization for results of Example 6;

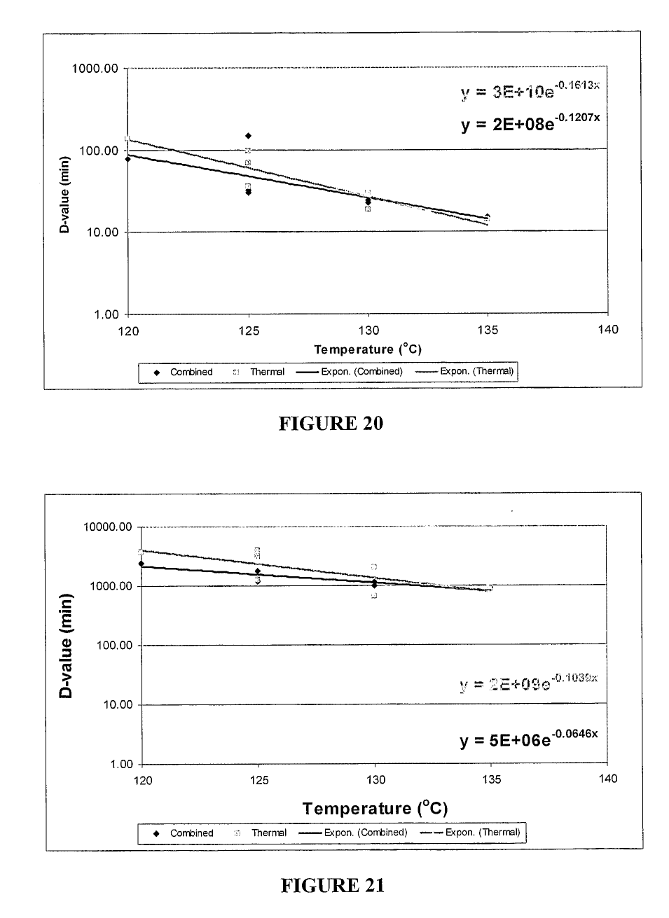

FIG. 20: a graph showing the Decimal reduction for Chroma value (C*) for results of Example 6;

FIG. 21: a graph showing the Decimal reduction for Luminence value (L*) for results of Example 6.

### DETAILED DESCRIPTION OF THE INVENTION

By way of background to assist understanding of the invention, when a gas is heated in an enclosure its pressure will rise according to ideal gas law at moderate pressures, or according to well-known generalized correlations for high pressure. Gases are compressible and hence the pressure increase will be very limited. When liquid is heated it will expand according to its thermal expansion coefficient, which is a function of temperature (and to a lesser extent of pressure). The thermal expansion coefficient of most liquids is significantly higher than those of metals; hence any heated liquid contained in for example a metallic container will undergo positive expansion. If thermal expansion of the liquid is constrained, pressure within the constrained system will rise significantly as the liquid is heated. The thermal expansion coefficient and the compressibility of the liquid will determine the pressure rise, as shown by the following equations:

$${( \frac{\partial P}{\partial T} )}_{v} = \frac{β}{κ}$$

where β and κ are the thermal expansion of the liquid (1/K) and its compressibility (1/bar). Hence liquids with high thermal expansion and low compressibility (such as glycerine) will generate the highest pressure when heated in enclosure.

Where an expansion medium is included within the confined volume to generate or contribute on heating to pressure generation on the product, the medium may optionally be a medium which will undergo a phase change and a volume increase on phase change such as a wax or a similar medium which is a solid at room temperature but melts at the processing temperature accompanied by a volume increase.

FIG. 1 schematically illustrates one implementation of the method of the invention. The “article” to be processed, includes liquid products such as juice, milk, or honey for example, a paste such as tomato paste, or any other pumpable food or non-food product is pumped into tubes 1 of a heat exchanger 2, from a bulk supply schematically illustrated at 4 (or typically alternatively an upstream stage in a food processing plant) by a food grade pump 3, to completely fill the heat exchanger tubes (absent of any air or other gas). When the heat exchanger tubes have been filled with the product, inlet and outlet valves 5a and 5b (the latter open to allow the escape of any air), which may be manual or solenoid-controlled valves for example, are closed to isolate the product within the heat exchanger. Hot water, steam, oil, or other heating medium is circulated in the outer jacket around the tubes 1 of the heat exchanger containing the product, and heats the product within the heat exchanger tubes 1 to a predetermined temperature. Heating medium inlet and outlet 7a and 7b to the heat exchanger 2 outer jacket are schematically illustrated as shown. The product resident within the heat exchanger tubes 1 is heated causing expansion of the product within the confined volume of the heat exchanger tubes (with inlet and outlet valves 5a and 5b closed), so that the product is subjected to a pressure increase within the heat exchanger, which combined with the elevated temperature, is sufficient to sterilise or pasteurise the product. The temperature to which the product is heated is selected to sterilise or pasteurise the product, with minimum damage to the quality of the product where the product is a food product for example. Subsequently valves 5a and 5b are opened and the product is pumped from the heat exchanger, allowing the second batch of fresh product to enter for treatment. It has been found that typically heating a liquid food product to a temperature approaching 100° C. will generate pressure of about 700 bar. With a treatment time of about 90 minutes a 4-log reduction of the B. stearothermophilus may be achieved. Thermal sterilization at ambient pressure cannot be achieved at 100° C. as the log reduction will be very small. At temperatures above 100° C., the log-reduction of the B. stearothermophilus has increase by factor of 5 to 10 by applying the method of this invention as shown in FIG. 14, which includes measurements conducted at high temperatures. At temperatures below 100° C., the difference in the log-reduction between the two types of treatment is even bigger

In an alternative form of the method of the invention the heating medium may be circulated in the outer jacket around the heater exchanger tubes continuously, and at each operation the product inlet and outlet valves 5a and 5b are opened, and a new batch or volume of product to be sterilized or pasteurized is pumped rapidly into the tubes of the heat exchanger, while at the same time the previously processed batch is pumped from the heat exchanger. The pumping must be very rapid so that during pumping of the product into the heat exchanger the product does not undergo significant heating or thermal expansion before the inlet and outlet valves are closed, but substantially all or at least a major part of the heating and thermal expansion of the product occurs after closing of the inlet and outlet valves.

In one particular alternative form the heat exchanger tubes may pass through a solar collector so that the heat is derived wholly or at least in part from solar energy, to provide what may be referred to as a solar pasteurizer/sterilizer. A solar collector may have internal tubes through which a liquid or paste product is pumped in batches, and then confined via closing of inlet and outlet valves, with each batch being retained within the tubes within the solar collector for a period of time sufficient to pasteurize or sterilize the product via heating and thermal expansion. Alternatively, again the heat exchanger may be of a conventional form, but the heating medium which is pumped through the tubes of a heat exchanger of the general type shown in FIG. 1 for example, may be heated via a conventional solar collector. The solar pasteurizer may be in flat plate, CPC or parabolic trough form. In this invention, microwave may also be applied as a source of heating.

FIG. 2 schematically illustrates another implementation of the method of the invention. A product F which may for example be a solid food product such as meat or seafood packed in an evacuated plastic pouch for example, or alternatively a liquid or paste food product in an evacuated plastic pouch, or a non-food product, is placed within vessel or chamber 10 connected to heat exchanger 2, through a door (not shown) in vessel or chamber 10. With valves 5a and 5b open a liquid which may be simply water for example or possibly a medium with a higher thermal expansion co-efficient, is pumped into the interior of the heat exchanger and the chamber 10 connected to the heat exchanger to completely fill the heat exchanger and the chamber 10 around the product F (any air or other gas is evacuated or expelled). Valves 5a and 5b are then closed. Hot water is circulated within the outer jacket of the heat exchanger to heat the high expansion liquid medium within the heat exchanger and chamber 10, generating pressure within the heat exchanger and chamber 10 on the product F, which is sufficient when combined with the elevated temperature to sterilise or pasteurise the product. The treatment chamber may optionally also be heated via for example another surrounding water jacket, resistance heating or microwave heating. Subsequently valves 5a and 5b are opened to release the pressure so that the product F may be removed by opening the chamber 10 (details not shown).

FIG. 3 schematically illustrates a further implementation of the invention. During processing the product to be treated is contained in a chamber 11 connected to the interior of heat exchanger 2 via a diaphragm 12 as shown. The product may be a liquid or paste product which is pumped into the chamber 11 to fill the chamber, via a pump 3 from a supply schematically indicated at 4, with valves 6a and 6b open. Alternatively the product may be a solid or liquid evacuated pouch-packed product for example, which is placed into the chamber 11 via a door (not shown), before liquid is pumped into the chamber 11 to fill the chamber 11 around the product. An expansion medium in the heat exchanger 2 as before is heated by circulating hot water/or oil through the outer jacket of the heat exchanger, to cause the medium within the tubes of the heat exchanger to expand and deflect diaphragm 12 into the chamber 11 to apply pressure to the product being treated within the chamber 11. In FIG. 3 the diaphragm is schematically shown distended into the chamber 11 during heating. At the same time the chamber 11 may be separately heated, but where the product being treated is a food product, typically to a lower temperature. For example water or oil may be circulated in a jacket around the chamber 11 or the chamber 11 may be heated via a resistance heating or microwave heating or similar. After processing the treated product is removed from the chamber 11. For example valve 6b may be opened and the product pumped from the chamber while valve 6a remains closed. Valve 6b may then be closed and valve 6a opened to allow the next batch of product to be delivered into the chamber 11.

This configuration enables the expansion medium to be heated to a higher temperature then for example 100° C., to maximise thermal expansion of the expansion medium and pressure on the product, without at the same time elevating the temperature to which the product is subjected to the same higher temperature, so that the temperature to which for example a food product is subjected can be kept below normal sterilization temperatures.

In a further implementation an expansion medium having a high thermal co-efficient of expansion may be contained within smaller spheres, particles or tubes of an elastic material which are distributed through a liquid product. When the product is heated the high expansion particles expand more than the product being processed, increasing pressure on the product. As the processed product is pumped from the heat exchanger the high expansion particles may be filtered or sieved from the food product, for re-use. Alternatively they may be left in the system to cool for the next batch.

The method of the invention may be implemented in a batch processing system or alternatively in a semi-continuous system. For example in a semi-continuous processing system utilising a heat exchanger arrangement similar to that of FIG. 1, a heating medium may be continuously circulated within the outer jacket of the heat exchanger, and liquid or paste product pumped into the heat exchanger, isolated within the heat exchanger for the period of time required to heat and pressurise and thereby sterilise or pasteurise the food product, and then valves 5 or the equivalent opened to enable pumping in of the next batch of product while removing to the next stage of a processing line the product batch just processed, and so forth.

In a variation of the method particularly applicable to the processing of liquids such as juices, or beverages to be carbonated for example, liquid CO₂ may be mixed with the liquid product to be processed, under pressure which will maintain the CO₂ in the liquid phase. The liquid product and liquid CO₂ are pumped into the treatment vessel or heat exchanger under such pressure, and subjected to pressure assisted thermal sterilisation or pasteurisation as described above. The presence of the CO₂ increases inactivation of bacterial spores present and reduce the pressure needed for effective sterilization. The liquid product after processing may be pumped out to atmospheric pressure, and a system may be provided for recycling the CO₂ by capturing the CO₂ gas and compressing it back to liquid form for reuse. An advantage may be that the phase change of the CO₂ from liquid gas on exit of the CO₂ to atmospheric pressure will assist in more rapidly cooling the product after processing, reducing any heat deterioration of the product which may otherwise occur

The invention is further illustrated by the following examples:

### Example 1

FIGS. 4 and 5 show the pressure increase achieved when different liquids were heated in an experimental vessel up to a temperature of 95° C. This vessel is illustrated in FIG. 11. It has a central pressure vessel 40 contained within a water bath 41 having a lid 42 with a key hole. The upper and lower section of the vessel are welded (with a weld 43) to provide an enclosure with very thick wall. At the top hole a pressure transducer 45 was connected to measure the pressure in the unit, while a plug 44 was used to plug the bottom opening used to drain the treated liquid.

The liquids were contained in a pressure vessel which was in turn heated in a water bath. Glycerine provided the maximum pressure. The main criteria for selecting fluids to generate pressure are safety, low compressibility and high thermal expansion. FIG. 6 shows the increase in the pressure due to the heating of different charges of a paraffin wax (RT20) in an experimental vessel. The large volume increase due to melting is the cause of the high pressure generated. The figure also shows that the high pressure increases the temperature at which the wax melts from in the range 18-22° C. to up to 60° C. (depending on pressure). The melting region is well characterized by the steeper slope of the pressure-time relationship. After complete melting the rate of increase in pressure becomes less. This phenomenon applies well to any liquid such as water which freezes at temperatures well below 0° C. under high pressure.

### Example 2

The effectiveness of the method of the invention for deactivating in particular B. stearothermophilus was experimentally tested. It has been noted throughout the literature that B. stearothermophilus (a very heat resistant bacterial spore) cannot be inactivated by high pressure alone. If B. stearothermophilus is completely deactivated the food may be assumed to be completely sterilized.

There is a particular temperature for bacterial spores to activate into a germinative form. For B. stearothermophilus range of about 49°-55° C. is generally proposed (Lund, 1975). Then moderate pressure will be sufficient to kill the germinated spores. Pressures of only a few hundred atmospheres could still cause germination but would be too low to cause inactivation. Therefore, many researchers have carried out experiments at Ultra High Pressures (beyond 100 MPa) combined with high temperatures. All such experiments primarily utilised external pressure, unlike in this invention in which the pressure is generated thermally, so the heating is utilised for two purposes; pressure generation and thermal killing.

We have studied the effectiveness of the method of the present invention in the inactivation of B. stearothermophilus spores in water heated at constant volume to various temperatures. The water was contained in a pressure vessel [FIG. 11], which was in turn heated in a water bath, as in Example 1 above. The results of combined temperature/pressure treatment at 90° C., 100° C. and 110° C., with their corresponding generated pressures are shown in FIGS. 8 to 10. Thermal treatment alone showed no reduction in the B. stearothermophilus spores count, while when the water is heated in the vessel more than 6-log reduction was observed. The pressure achieved by the method of this invention together with the mild thermal treatment caused such high inactivation rate at low temperature minimizing damage to nutrient in food.

### Example 3

Experimental work was carried out with a test rig as shown in FIGS. 11 and 12. The test rig comprised a part 15 simulating a single tube of a tube heat exchanger of the general type shown in FIG. 1, comprising an inner tube 16 for containing during processing the product to be pasteurised or sterilised, and an outer shell or jacket 17 through which the heating medium was circulated to heat the product within the inner tube 16. The heating medium was circulated within the outer jacket 17 via inlet and outlet ports 18. The product was pumped into the heat exchanger tube 16 via pump 19, through hand controlled inlet and outlet valves 20 and 21.

Liquid was pumped into the inner tube 16 with the valves 20 and 21 open, and then the valves 20 and 21 were closed, to contain the liquid within the confined volume of tube 16. Hot water as a heating medium was then circulated within the outer shell 17 via ports 18 to subject the liquid within the confined valve of the inner tube 16 to the pressure assisted thermal processing of the invention. This arrangement is made to replicate, as close as possible, the suggested industrial configuration shown in FIG. 1 of this invention. FIG. 7 shows the equilibrium pressure-temperature relationship for water in the inner tube of the heat exchanger, while FIG. 13 shows the transient temperature and pressure in the tube.

### Example 4

We have also conducted experimental investigations of the invention at temperatures above 100° C. In order to do so the equipment used in Example 3 had to be modified so that it can be operated at temperatures above 100° C. In-can sterilization is usually done at 121° C. while a product such as UHT milk is treated at 140-150° C. for very short time.

The water as a heating fluid was replaced by oil. But because the viscosity of the oil is significantly higher than that of water, it was necessary to increase the diameter of the external pipe of the heat exchanger to allow larger annular area for the oil to flow easily. This leads to faster heating, which is needed for the high temperature-short time treatment. Further improvement is possible in industrial size units

The inner tube containing the treatment fluid extends beyond the treatment zone, which may cause some contamination during sampling, especially when high reduction in bacteria count is to be achieved. It was decided to surround that section of the tube with a heating tap to sterilize the tube prior to each experiment. Care was taken to allow the tube to cool before starting the experiment and certainly before withdrawing the sample to avoid extra inactivation, which could be caused by the electric heating.

The same experimental procedure was used with the exception that the outlet section of the heat exchanger tube had to be sterilized thermally (because it is outside the treatment zone) using electrical heating tape controlled by a temperature controller. FIG. 14 shows a comparison between the experimental measurements of inactivation of B. Stearothermophilus using the equipment of this invention and those usually obtained from thermal sterilization. The data for thermal sterilization contains also some of our measurements which are in a good agreement with those reported in the literature. Thermal sterilization experiments are usually conducted in capillary tube so that the liquid in it reach treatment temperature very fast and uniformly. In the equipment constructed based on this invention, an effective treatment temperature was calculated and fotnd significantly lower than the final treatment temperature when the treatment time is short. This effective temperature (Teff) is calculated as follows:

$${k}_{eff} = \frac{{\int }_{0}^{t} k d t}{{\int }_{0}^{t} d t}$$

$${k}_{eff} = A {e}^{- \frac{E}{{RT}_{eff}}} = \frac{A}{t} {\int }_{0}^{t} {e}^{- \frac{E}{RT}} d t$$

$${T}_{eff} = \frac{- \frac{E}{R}}{ln [ \frac{{\int }_{0}^{t} {e}^{- \frac{E}{RT}} d t}{t} ]}$$

Where T=F (t), which is known from the measurements as shown in FIG. 13. The integral term was calculated using Simpson's Rule of Integration. In the above equations, “t” is treatment time, “R” is gas constant and “E” is the Activation Energy of spore destruction.

The treatment method of this invention provided significantly higher inactivation than that of thermal sterilization, especially at low temperature. This is clearly shown in FIG. 14, where all the points are lying well above the 45′-line. This suggests that the sterilization method suggested in this invention can be done at lower temperatures to minimize damage to nutrient in food. FIG. 15 is another way of presenting the data. It is common to use the D value as a measure of how easy or difficult to inactivate micro organisms. It is defined as the time, in minutes, needed to cause one logarithmic reduction in the bacterial count. Significant reduction in the D value was achieved by employing the pressure assisted thermal sterilization, suggesting that there is a great benefit of applying the treatment of this invention.

FIG. 16 is a plot of few experiments conducted on milk using both thermal and pressure assisted thermal sterilization. The decimal reduction time of B. Stearotheimophilus in milk is higher than that in water. Similar to what has been observed in water, there is a significant reduction in the value of D when the method of this invention using pressure assisted sterilization is applied.

### Example 5

FIG. 17 is a plot conducted using the same equipment described above but for water containing B. cereus spores. Some of the points shown in the figure are average of large number of measurements. All the points lying above the 45-degree line indicating that the pressure assisted sterilization has enhanced the inactivation rate. However, the enhancement is not as high as what has been observed for B. stearothermophilus.

### Example 6

Both thermal and pressure assisted thermal sterilization were applied using milk as a model food. The objective was to test the change in the colour of the milk due to both treatments. The experimental procedure was the same as that used for inactivation of micro organisms.

L* represents the luminance of colour (i.e. lightness, indicating black when L*=0, and white when L*=100), “a*” represents the position between red (+) and green (−), and “b*” represents the position between yellow (+) and blue (−). The Chroma value (C*) which describes the degree of saturation, purity or intensity of colour (Kwok, MacDougall and Niranjian, 1998). It can be calculated by the following expression:

C*=\sqrt{a*²+b*²}

Increasing the value of C* is an indication of milk browning, while decrease in L* is an indication of loss of milk whiteness. FIGS. 18 and 19 show how the values of C* and L* of milk change with treatment time and temperature. Milk discoloration starts at L*=95.55 (≈1% change in L*) as shown in FIG. 20 defined by the region below the horizontal line, which is in agreement with the information reported in the literature for milk (Tetra Pack, 1995). Browning is evident from the changes in the value of C* but milk is usually treated for very short time to avoid that. The objective of showing such severe treatment is to illustrate that there is no extra discoloration of milk caused by the pressure generated thermally. This is even more clear from the D values of C* and L* shown in FIGS. 20 and 21. There is no decrease in the values of D due to pressure and it may be concluded that the D value of colour is temperature dependant but pressure independent, unlike microbial inactivation. Based on these results which are supported by the literature, pressure assisted sterilization/pasteurization will improve the efficiency of microbial inactivation without significant change to food properties such as colour. Small molecules such as vitamins are not influenced by pressure but are sensitive to temperature, so they could be retained better if the sterilization temperature is lowered by the method of this invention. Overall the D value of colour parameters and probably most other parameters defining milk quality are much larger than the D values of the most thermal resistance spores such as B. Stearothermophilus.

### ADVANTAGES OF THE INVENTION AND INDUSTRIAL APPLICATION

In summary, as previously indicated an advantage of one possible method of the invention when used for pasteurising or sterilising food products in particular is that it is possible to pasteurise or sterilise products at relatively low temperatures or shorter treatment time, reducing the likelihood of thermal damage to vitamins or other nutrients in the food products which may otherwise occur at higher temperatures. The pressure, by the method of this invention, is generated from the heat needed for the thermal treatment eliminating the need for a high pressure pump. Examples of specific applications of an embodiment of the invention may be
Milk is usually pasteurised at 72° C. for 15 seconds. This treatment kills all pathogens and also cause minimum changes in milk quality (colour, protein content, vitamins content, etc.). However spores are not affected by such treatment and hence the shelf life is limited to only few days. In-can sterilization is a process used to sterilize food canned products, including milk for long shelf life. For milk, treatment is usually done at 121° C. for 15-20 minutes. At such sever thermal treatment significant amount of vitamins and other nutrients are destroyed. The milk undergoes also significant undesirable taste and colour changes. UHT treatment of milk is usually done at 135° C. for 15 seconds. This is a sterilization process employed as alternative to in-can sterilization as it destroys spores as well as vegetative micro organisms. Under such treatment, 10-12 log reduction in mesophilic spores such as B. Subtilus and 8 log reductions in B. Stearothermophilus is expected. Under such condition, some quality change in milk occurs but milk quality is by far better than milk sterilized in cans. Efforts are still being made to improve UHT-milk to reach a flavour closer to ordinary pasteurized milk (Dairy Processing Handbook).

By applying the method of this invention, it is possible to reduce the damaging effect of thermal treatment on food by reducing the operating temperature or time with the assistance of the mild pressure generated from heating. This will produce sterilize milk but with less changes in taste, colour and vitamins content since these quality attributes are sensitive to temperature and not pressure.
Yoghurt manufacture, where temperatures in the range of 95-99 degrees C. for about 10 minutes or 85 degrees C. for about thirty minutes are typically used to kill pathogens and inactivate enzymes to increase the shelf life of the yoghurt. Again the process of the invention may enable pasteurization or sterilization to be carried out at a lower temperature and/or for a shorter time.
In the pasteurization of tomato paste, which is typically carried out at 109 degrees C. for about 2.25 minutes or 96 degrees C. for about 3 minutes in which it is important to avoid browning. Pasteurization of the paste at a lower temperature which will reduce the risk of browning is likely to be effective by the method of the invention.
High pulp content juices, which require strong thermal treatment for pasteurization, of the order of 90 degrees C. for 60 seconds typically, because enzymes tend to concentrate in pulp rather than the juice itself. Using the method of the invention pasteurization will be achievable at a lower temperature, resulting in less destruction of vitamin C. Our results indicated that there is no effect of mild pressure on enzyme.
The method of the invention may also find application in the production of sweet acidophilus milk. Where again acceptable pasteurization is likely to be achievable at lower temperatures and/or with shorter processing times than conventionally used.
The method of the invention may also find application in the treatment of solid food products such as meat, seafood such as shellfish, and some fruits, for example.

The foregoing describes the invention including specific implementations thereof. Alterations and modifications as will be obvious to those skilled in the art are intended to be incorporated within the scope hereof.

### REFERENCES

Ananta, Edwin. Heinz. Volker, Schluter. Oliver, and Knorr, Dietrich; 2001., Kinetic studies on high-pressure inactivation of Bacillus stearothermophilus spores suspended in food matrices, Innovative Food Science & Emerging Technologies 2 (4SU) 261-272.
Furukawa. S, & Hayakawa, (2000); Investigation of desirable hydrostatic pressure required to sterilize Bacillus stearothermophilus IFO 12550 spores and its sterilization properties in glucose, sodium chloride and ethanol solutions, Food Research International 33 (2000) 901-905.
Gosta, B. Dairy processing handbook: Tetra Pak Processing Systems AB, 1995. Gould, G. W, The microbe as a High Pressure target Chapter—3, High pressure processing of foods/D. A. Leeward . . . [et al.]. Nottingham University Press, 1995
Hayakawa, I., Kanno, T., Tomita, M. and Fujio, Y. 1994a. Application of high pressure for spore inactivation and protein denaturation. J Food Sci. 59(1):159-163
Ledward. D. A, High pressure processing—the Potential chapter—1, High pressure processing of foods/D. A. Ledward . . . [et al.]. Nottingham University Press, 1995
Metrick, C., Hoover, D. G., and Farkas, D. F. 1989. Effects of high hydrostatic pressure on heat-resistant and heat-sensitive strains of Salmonella. Journal of Food Science 54(6): 1547-1549, 1564.
Rademacher B, Pfeiffer. B and Kessler. G. H, Inactivation of Micro organisms and Enzymes in Pressure-Treated Raw Milk, High Pressure Food Science, Bioscience and Chemistry, Ed by Isaacs. N. S., The Royal Society of Chemistry 1998 pages 145-151.
Raso, J., and Barbosa-Canovas, G. V. 2003. Nonthermal preservation of foods using combined processing techniques. Critical Reviews in Food Science and Nutrition 43(3): 265-285.
Tholazan, J. L, et. al. Physiological Responses and Morphological Changes of Salmonella typhimuriumand listeria monocytogenes to high hydrostatic pressure Treatments, High Pressure Food Science, Bioscience and Chemistry, Ed by Isaacs. N. S., The Royal Society of Chemistry 1998 pages 442-447.
Kwok, K. C., MacDougall, D. B. & Niranjan, K. (1998) Journal of Food Engineering, 40, 15-20.
Tetra Pak, (1995) Dairy Processing Handbook, Chapter 9, 215-232.

1. A method for pasteurising or sterilising an article, comprising the step of heating the article and/or a medium associated or in communication with the article within a confined volume sufficiently that expansion of the article and/or of the medium subjects the article to a pressure sufficient when combined with the elevated temperature to pasteurise or sterilise the article.

2. A method as claimed in claim 1 comprising heating the article to an elevated temperature which is less than that required to pasteurise or sterilise the article in the absence of the pressure.

3. A method as claimed in claim 1 comprising heating the article to the elevated temperature for a duration of exposure of the article to the elevated temperature less than that required to pasteurise of sterilise the article in the absence of the pressure.

4. A method as claimed in claim 1 comprising heating the article to an elevated temperature and for a duration of exposure of the article to the elevated temperature, less than that required to pasteurise of sterilise the article in the absence of the pressure.

5. A method as claimed in any one of the preceding claims wherein the article is a food product.

6. A method as claimed in claim 5 comprising heating the article to a temperature below about 140° C.

7. A method as claimed in claim 6 comprising heating the article to a temperature below about 120° C.

8. A method as claimed in claim 5 comprising heating the article to a temperature effective to sterilise the article in the absence of the pressure/at ambient pressure, but for a shorter time.

9. A method as claimed in any one of claims 5 to 8 wherein the article is in liquid form.

10. A method as claimed in any one of claims 5 to 8 wherein the article is in a semi-liquid form.

11. A method as claimed in any one of claims 5 to 8 wherein the article is in a solid form.

12. A method as claimed in any one of claims 1 to 4 wherein the article is selected from the group consisting of a pharmaceutical product, a medical product and a biological material.

13. A method as claimed in any one of the preceding claims wherein the medium is present and has a co-efficient of thermal expansion higher than that of the article being processed, the method comprising heating the medium to generate or increase pressure on the article such that the pressure is sufficient, when combined with the elevated temperature, to pasteurise or sterilise the article.

14. A method as claimed in claim 13 wherein the article is in liquid or semi liquid form and the expansion medium is distributed throughout the article.

15. A method as claimed in claim 13 comprising containing the article within a first part of the confined volume and containing the expansion medium within a second part of the confined volume separated by a diaphragm or other means effective to transfer pressure induced by expansion of the expansion medium to the article being pasteurised or sterilised.

16. A method as claimed in any one of the preceding claims wherein heating is achieved through one or more of isolating the article within a heat exchanger, heating of a chamber within which the article is contained by electrical resistance, by microwave heating, by solar or waste heat.

17. A method as claimed in any one of the preceding claims wherein the sterilisation or pasteurisation of food products is applied at a stage in a food processing plant to treat the food product prior to packaging.

18. An article pasteurised or sterilised according to the method as claimed in any one of claims 1 to 17.

19. Apparatus for sterilising or pasteurising an article, including a processing chamber providing a confined volume for containing an article to be processed, heating means for heating the article, and wherein the processing chamber is configured such that heating the article and/or an expansion medium associated or in communication with the article generates a pressure applied to the article sufficient when combined with the elevated temperature to pasteurise or sterilise the article.

20. Apparatus as claimed in claim 19 including a control system arranged to heat the article sufficiently that expansion of the article and/or of the expansion medium subjects the article to a pressure sufficient when combined with the elevated temperature to pasteurise or sterilise the article.

21. Apparatus as claimed in claim 19 or 20 wherein the processing chamber includes a first part for containing the article to be processed and a second part for containing the expansion medium, the second part being separated from the first part by a diaphragm or other means effective to transfer pressure induced by expansion of the expansion medium to the article being processed.

22. Apparatus as claimed in any one of claims 19 to 21 wherein the heating means is selected from the group consisting of a heat exchanger, an electrical resistor, a microwave source, a solar heating source or a waste heating source.

23. Apparatus as claimed in any one of claims 19 to 22 wherein the article to be processed is a food product and the apparatus is situated in a food processing plant to treat the food product prior to packaging.

24. A method for pasteurising or sterilising a food product, comprising the step of heating the food product and/or a medium associated or in communication with the food product within a confined volume sufficiently that expansion of the food product and/or of the medium subjects the food product to a pressure sufficient when combined with the elevated temperature to pasteurise or sterilised the food product.

25. A method as claimed in claim 24 comprising heating the food product to a temperature below about 120° C.

26. A method as claimed in claim 24 or 25 wherein the food product is in liquid or semi liquid form.

27. A method as claimed in claim 26 wherein the medium is present and has a co-efficient of thermal expansion higher than the food product and is distributed throughout the food product, the method comprising or including heating the medium to generate or increase the pressure such that the pressure is sufficient, when combined with the elevated temperature, to pasteurise or sterilise the food product.

28. A method for sterilising or pasteurising an article substantially as herein described with reference to any one or more of the examples or drawings.

29. Apparatus for sterilising or pasteurising an article substantially as herein described with reference to any one or more of the examples or drawings.
